(19) United States
(12) Patent Application Publication
(10) Pub. No.: US 2011/0184328 A1
(43) Pub. Date: Jul. 28, 2011

(54) JOINT PAIN TREATMENT BANDAGE

(75) Inventors: Claude Chieze, Prevessin-Moens (FR); Jocob Zijlstra, Coppet (CH)

(73) Assignee: NOVARTIS AG, Basel (CH)

(21) Appl. No.: 13/061,799

(22) PCT Filed: Sep. 24, 2009
(86) PCT No.: PCT/EP09/62356

§ 371 Date: Mar. 2, 2011

(30) Foreign Application Priority Data
Sep. 26, 2008 (EP) 08165250.5

(51) Int. Cl. A61F 13/00

(52) U.S. Cl. 602/48

(57) ABSTRACT

The invention relates to a medical bandage, which comprises at least two transdermal patches, and wherein said at least two transdermal patches each comprise a pain-relieving pharmaceutically active substance such as an NSAID.

The invention concerns the topical administration of pharmaceutically active substances for the treatment of pain, especially such occurring in limbs or joints of a patient, e.g. ankle sprain.

Typically, pain of this kind is severe so that the current standard treatment is systemic, mainly oral, treatment with analgesics and anti-inflammatory drugs, such as NSAIDs (non-steroidal anti-inflammatory drugs) and COX-2 inhibitors. The side effects of the latter treatment have been the subject of many discussions, especially gastrointestinal and cardiovascular adverse effects. It is an object of the present invention to avoid systemic treatment of joint pain by administering pain-relieving pharmaceutically active substances topically but nevertheless providing pain relief comparable to that of systemic treatment.

Currently, said topical treatment is typically done by applying semi-solid topical dosage forms, like gels, emulsion-gels or creams, to the site of pain. The limitations of said treatment are well known and mainly concern the limited period of time during which the active substance can effectively permeate, i.a. due to rubbing, washing and sweating effects. An improvement in this regard are transdermal patches comprising pharmaceutically active substances in a more occlusive manner, thus ensuring a longer permeation of drug substance.

Therefore, currently transdermal patches comprising a pharmaceutically active substance, in particular an NSAID, are applied to aching limbs or joints of a patient. Very often, however, the benefit of said transdermal patches on the joints (articulations) is reduced. This is mainly due to movements and changes of volumes due to swelling, often resulting in patch detachments.

Therefore it is an object of the present invention to provide a topical dosage form comprising a pharmaceutically active substance, which topical dosage form ensures that said pharmaceutically active substance is tightly fixed in place (at the site of pain) and is correctly distributed to allow it to penetrate through the skin over a long period of time.

It is another object of the invention to, in addition to the drug treatment, stabilize the aching limb or joint by exerting a fixing, guiding, supporting and/or assisting function on the aching limb or joint.

It is a further object of the invention to keep the site of pain warm because warmth, or a warm feeling respectively, will add to pain relief in e.g. a painful joint.

The various objects of the present invention are met by providing an “active” medical bandage, which means that one or more pharmaceutically active substances, preferably one, are incorporated into a medical bandage. Thereby, the patient will benefit from the beneficial effects of the active substances as well as from the stabilizing functions of a bandage at the same time. Moreover, the bandage of the invention will serve to keep the joint warm thus contributing to reduce pain, and therefore altogether improve the patient condition.

In other words, a medical bandage is sought that simultaneously provides treatment with the active substance, support and warmth, each of which being useful for a good, painless and fast recovery from an aching limb or joint, e.g. ankle sprain. The combination of said three features will enable patients to come back to normal life faster.

It turned out during the course of experimentations that directly impregnating the medical bandage with active substance was not a technically feasible option because the characteristics of a good medical bandage were too different from the characteristics of a good, reliable, controlled support for drug delivery. Additionally, it is an integral part of the present invention to provide the active substances at specific locations, namely there where they will be most useful, rather than allowing drug permeation from the entire bandage application surface.

Therefore, the invention relates to a medical bandage, which comprises at least two transdermal patches, wherein the at least two transdermal patches are attached to the inside of said medical bandage thus getting into contact with the patient's skin upon application, and wherein said at least two transdermal patches each comprise a pain-relieving pharmaceutically active substance.

In a particular embodiment of the invention, the inside of said medical bandage (i.e. the inner surface thereof) is adhesive to the skin. Said inside can be laminated with a topically acceptable, typically non-irritant, adhesive, e.g. a polyacrylic adhesive, a silicone adhesive, an EVA adhesive or a block elastomer such as SEBS, SIS or SBS. Typically, said adhesive is selected such that it allows for replacement of the bandage during treatment. As a result, the initial intended fixation of the medical bandage will be even more reliable, which can in general be desirable but may be of particular importance for joints (or limbs) having substantial, important muscles (with the potential to cause the medical bandage to move in an undesired manner), such as knee or shoulder.

In more detail, it is, for example, possible to cover the entire inside of the medical bandage with an adhesive, in which case either the adhesive material has to be selected such that it is compatible with the materials of the transdermal patches, e.g. by using EVA or SIS as adhesive, or the materials of the transdermal patches have to be isolated from the adhesive, e.g. by attaching the transdermal patches via an impermeable backing layer to the bandage.

An alternative option is to cover the entire inside of the medical bandage with an adhesive but to omit those areas of the inside of said medical bandage where the transdermal patches will be placed. In that case, the transdermal patches can be attached to the bandage without impermeable backing layer if desired, and nevertheless a broader range of adhesive materials can be used, e.g. said topically acceptable, typically non-irritant, adhesives listed above.

In another embodiment of the invention, the medical bandage includes instructions, e.g. in the form of one or more drawings, on how to correctly apply said medical bandage to the intended joint or limb, which instructions are fixed, e.g. printed, on the outside of said medical bandage (i.e. the outer surface thereof).

Typically, said instructions will comprise indicating a starting point where to begin with the application of the medical bandage, which preferably is the position of the first patch relative to the intended joint or limb to be treated. Moreover, said instructions will typically further comprise information, preferably in the form of one or more drawings, as to how to progress with the application of the medical bandage and, optionally, also its final fixation (e.g. by clamps).

A pain-relieving pharmaceutically active substance is e.g. a non-steroidal anti-inflammatory drug (“NSAID”), or a local anesthetic, or a counter irritant, e.g. capsaicin.

Non-steroidal anti-inflammatory drugs are e.g. diclofenac, indomethacin, ibuprofen, piroxicam, acetylsalicylic acid (Aspirin®), naproxen, methyl salicylate, ketoprofen, tolfenamic acid, phenylbutazone, piroxicam, nimesulide, COX-2 inhibitors e.g. lumiracoxib, or omega-3 fatty acids e.g. those derived from fish oil, and pharmaceutically acceptable salts of any of said compounds. Preferred as NSAID is diclofenac or a pharmaceutically acceptable salt thereof. Especially preferred are diclofenac free acid, diclofenac sodium, diclofenac potassium, diclofenac diethylammonium and diclofenac epolamine, and in particular diclofenac sodium.

Local anesthetics are e.g. lidocaine, benzocaine, dibucaine or procaine.

The term “transdermal patches” is used herein in a broad sense. On the one hand, it includes transdermal patches as such, i.e. as commercially available and as broadly disclosed in the art.

Transdermal patches as such comprising pain-relieving pharmaceutically active substances are well known in the art. Typically, they are built up, in principle, by comprising an impermeable backing layer, one or more layers comprising the active substance (e.g. homogeneously distributed in a matrix or retained in a reservoir) and a protective layer which can be pulled off. The layer adjacent to the protective layer typically has pressure sensitive adhesive properties to ensure attachment of the patch to the skin. The impermeable backing layer of said transdermal patches as such can be glued to the medical bandage of the invention at the desired positions.

It is preferred to have one or more matrix layers (at least one of them) comprising the active substance. In that case, the matrix layer adjacent to the protective layer typically has pressure sensitive adhesive properties to ensure attachment of the patch to the skin.

On the other hand, however, the term “transdermal patches” is also used herein, preferably, for modified transdermal patches which are adapted to their specific use within the bandage. In said modified transdermal patches, the impermeable backing layer is lacking and the function of the latter is taken over by the medical bandage. Thus, in one embodiment of the invention, transdermal patches as such but without impermeable backing layer are fixed on the bandage in an analogous manner as they would conventionally be fixed on the impermeable backing layer.

In another embodiment of modified transdermal patches, the transdermal patches as such can be even further adapted to their intended use within bandages. A conventional transdermal patch as such is characterized in that typically there has to be ensured adhesion (a) between the (e.g. matrix) layer adjacent to the protective layer and the skin and (b) between the (e.g. matrix) layer adjacent to the impermeable backing layer and the impermeable backing layer. For the modified transdermal patches according to the present invention, (b) is still required, i.e. adhesion of the (e.g. matrix) layer to the bandage is accomplished e.g. either by inherent adhesiveness of the (e.g. matrix) layer or a separate adhesive layer. But (a) is not a “must” anymore for the modified transdermal patches because the fixation to the skin will be accomplished by the bandage anyway. In conclusion, in this embodiment of modified transdermal patches, the (e.g. matrix) layer adjacent to the protective layer can also be non-adhesive to the skin.

To reiterate: In principle, all transdermal patches comprising pain-relieving pharmaceutically active substances known in the art come into consideration for the present invention. Typically, the one or more (e.g.) matrix layers thereof (plus protective layer) are attached to a flexible bandage instead of an impermeable backing layer. Typically, before application of a medical bandage of the invention the individual protective layers (protection films) of each patch have to be removed.

Preferably, the invention relates to a medical bandage, which is intended for the treatment of an aching limb or joint of a patient, and wherein the at least two transdermal patches are positioned around said limb or joint such that upon application of said medical bandage the pain-relieving pharmaceutically active substance will penetrate into said limb or joint from at least two different spots close to the area that causes pain.

The specific setup of the medical bandages of the invention ensures that the transdermal patches will be tightened to the skin at the specific body locations that are closest to the respective tissues triggering the pain. Consequently, the active substance may reach the target tissues fast as well as a high concentration therein. Therefore, the efficacy in treating pain is high, and the onset of action is short.

Especially, the medical bandage of the invention is adapted to the treatment of ankle pain—e.g. ankle sprain—, wrist pain, knee pain—e.g. knee sprain or knee osteoarthritis—, elbow pain—e.g. tennis elbow or elbow osteoarthritis—, shoulder pain, or finger pain—e.g. finger sprain or finger osteoarthritis such as thumb osteoarthritis. Preferably, it is made of flexible tissue to adapt to all curves of the limb or joint involved, as e.g. ankle and foot in case of ankle sprain. In particular, the medical bandage of the invention is adapted to the treatment of ankle sprain(s).

By the tight fixation of at least two transdermal patches around the aching limb or joint, a large distribution of the pain-relieving pharmaceutically active substance all around said aching limb or joint is ensured, as e.g. in case of ankle sprain where a large distribution of the pain-relieving pharmaceutically active substance all around the ankle and supply of the latter to the ligaments and/or tendons are ensured.

By applying the medical bandage of the invention, the patient will benefit both from the active substance (for example an NSAID that will reduce pain and inflammation), and from the bandage that will support the joint (articulation) and maintain a warm temperature and reduce pain.

In general, the medical bandage of the invention can be applied to the aching limb or joint as long as desired, e.g. until pain is completely relieved. A convenient setting—but of course not the only one possible—is to replace it every 24 hours for a period of several days, e.g. 1 to 14 days, or 1 to 7 days, or 1 to 5 days, depending on the medical condition to be treated.

In a typical setting for treatment of e.g. ankle sprain, the medical bandage will be left in place for 24 hours, and then changed each day e.g. for a period of 5 days. Depending on the medical condition at the end of treatment, the medical bandage can also be replaced with a passive medical bandage, said “passive” medical bandage being defined as a medical bandage according to the invention but lacking any transdermal patches and thus any pain-relieving pharmaceutically active substance. A typical course of treating e.g. ankle sprain may include the application of e.g. one to five daily active medical bandages and one passive medical bandage whereby the latter may be applied longer than one day, e.g. for one to five days.

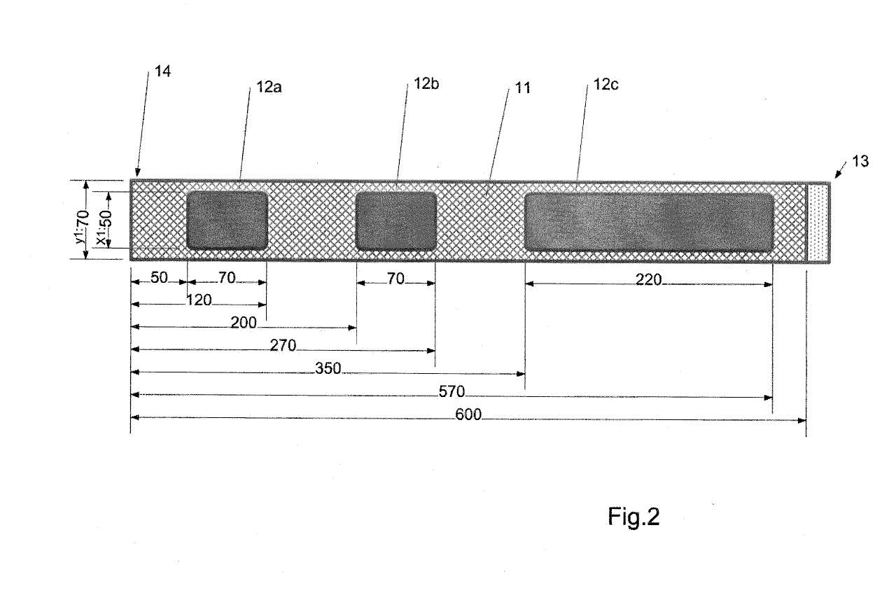

The number, size and positioning of the transdermal patches on the medical bandage are important so as to provide the aching limb or joint with sufficient drug substance penetrating. Corresponding information for various limbs and joints is provided in FIGS. 2-13 as examples. The broad terms “limb” and “joint” are intended to also cover the more specific parts thereof giving rise to experience of pain, e.g. corresponding ligaments, tendons or joint capsules.

Ankle: Preferably, the medical bandage has a length of 450-800 mm, a width of 40-100 mm, and comprises a transdermal patch for the internal ankle of 40-100 mm length and 30-90 mm width, a second transdermal patch for the external ankle of 40-100 mm length and 30-90 mm width and a third transdermal patch for the arch-of-the-foot ankle of 100-300 mm length and 30-90 mm width.

More preferably, the medical bandage has a length of 500-700 mm, a width of 60-80 mm, and comprises a transdermal patch for the internal ankle of 50-90 mm length and 40-70 mm width, a second transdermal patch for the external ankle of 50-90 mm length and 40-70 mm width and a third transdermal patch for the arch-of-the-foot ankle of 150-250 mm length and 40-70 mm width.

In particular, the medical bandage has a length of 600 mm, a width of 70 mm, and comprises a transdermal patch for the internal ankle of 70 mm length and 50 mm width, a second transdermal patch for the external ankle of 70 mm length and 50 mm width and a third transdermal patch for the arch-of-the-foot ankle of 220 mm length and 50 mm width.

Wrist: Preferably, the medical bandage has a length of 350-700 mm, a width of 40-100 mm, and comprises a transdermal patch for the ventral wrist of 30-80 mm length and 30-90 mm width, a second transdermal patch for the dorsal wrist of 30-80 mm length and 30-90 mm width.

More preferably, the medical bandage has a length of 400-600 mm, a width of 60-80 mm, and comprises a transdermal patch for the ventral wrist of 40-60 mm length and 40-70 mm width, a second transdermal patch for the dorsal wrist of 40-60 mm length and 40-70 mm width.

Elbow: Preferably, the medical bandage has a length of 500-800 mm, a width of 40-100 mm, and comprises a transdermal patch for the lateral epicondyle of 25-70 mm length and 30-90 mm width, a second transdermal patch for the oleocranon being a small patch as defined below, a third transdermal patch for the medial epicondyle of 25-70 mm length and 30-90 mm width, a fourth transdermal patch for the medial forearm being a small patch as defined below, and a fifth transdermal patch for the lateral forearm being a small patch as defined below.

More preferably, the medical bandage has a length of 600-700 mm, a width of 60-80 mm, and comprises a transdermal patch for the lateral epicondyle of 30-50 mm length and 40-70 mm width, a second transdermal patch for the oleocranon being a small patch as defined below, a third transdermal patch for the medial epicondyle of 30-50 mm length and 40-70 mm width, a fourth transdermal patch for the medial forearm being a small patch as defined below, and a fifth transdermal patch for the lateral forearm being a small patch as defined below.

Knee: Preferably, the medical bandage has a length of 500-1100 mm, a width of 40-100 mm, and comprises a transdermal patch for the medial patella of 20-50 mm length and 30-90 mm width, a second transdermal patch for the lateral patella of 20-50 mm length and 30-90 mm width, a third transdermal patch for the medial knee of 25-70 mm length and 30-90 mm width, and a fourth transdermal patch for the lateral knee of 25-70 mm length and 30-90 mm width.

More preferably, the medical bandage has a length of 600-900 mm, a width of 60-80 mm, and comprises a transdermal patch for the medial patella of 25-40 mm length and 40-70 mm width, a second transdermal patch for the lateral patella of 25-40 mm length and 40-70 mm width, a third transdermal patch for the medial knee of 30-50 mm length and 40-70 mm width, and a fourth transdermal patch for the lateral knee of 30-50 mm length and 40-70 mm width.

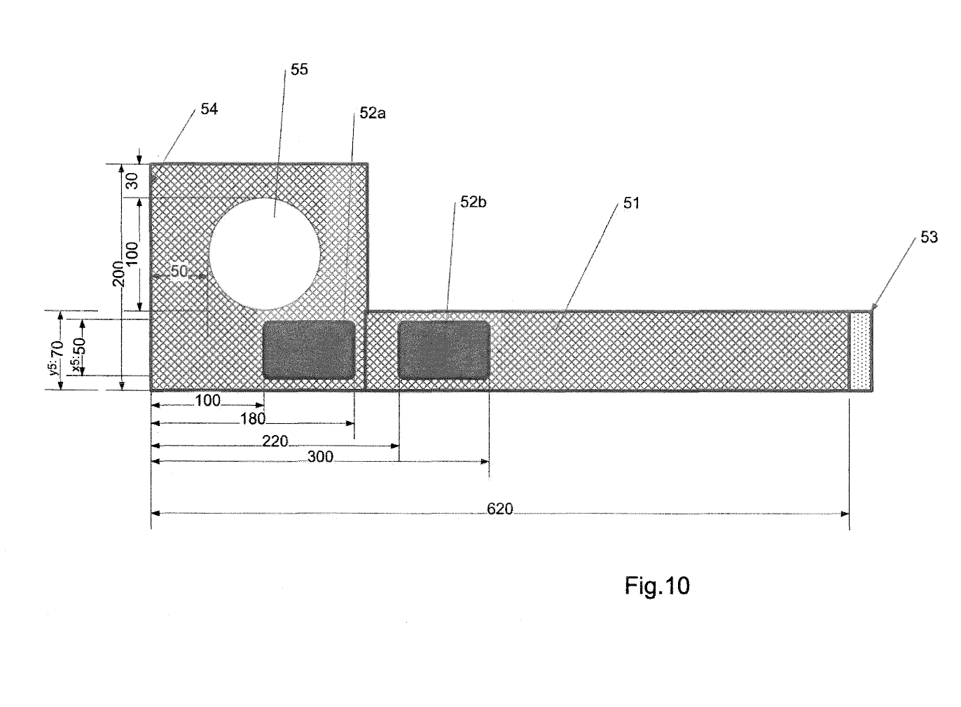

Shoulder: Preferably, the medical bandage has an overall length of 500-800 mm and consists of two parts which are firmly connected, namely (a) the “hole part” and the “bandage part” (as shown in FIG. 10). The hole part, preferably, has a length of 150-230 mm, a width of 170-230 mm, and comprises a hole which is circular and has a diameter of 70-150 mm. Said hole part further comprises a transdermal patch for the ventral shoulder of 50-110 mm length and 30-90 mm width. The connected “bandage part” has a length of 300-700 mm, a width of 40-100 mm, and comprises a transdermal patch for the dorsal shoulder of 50-110 mm length and 30-90 mm width.

More preferably, the medical bandage has an overall length of 550-700 mm and consists of two parts which are firmly connected, namely (a) the “hole part” and the “bandage part” (as shown in FIG. 10). The hole part, preferably, has a length of 170-210 mm, a width of 180-220 mm, and comprises a hole which is circular and has a diameter of 80-130 mm. Said hole part further comprises a transdermal patch for the ventral shoulder of 70-100 mm length and 40-70 mm width. The connected “bandage part” has a length of 350-550 mm, a width of 60-80 mm, and comprises a transdermal patch for the dorsal shoulder of 70-100 mm length and 40-70 mm width.

The invention further relates to a method of treating inflammatory diseases which comprises topically administering to a mammal in need of such treatment a therapeutically effective amount of one of the topical pharmaceutical compositions as specified herein above and below.

All dimensions indicated in the drawings are given in millimeters (mm).

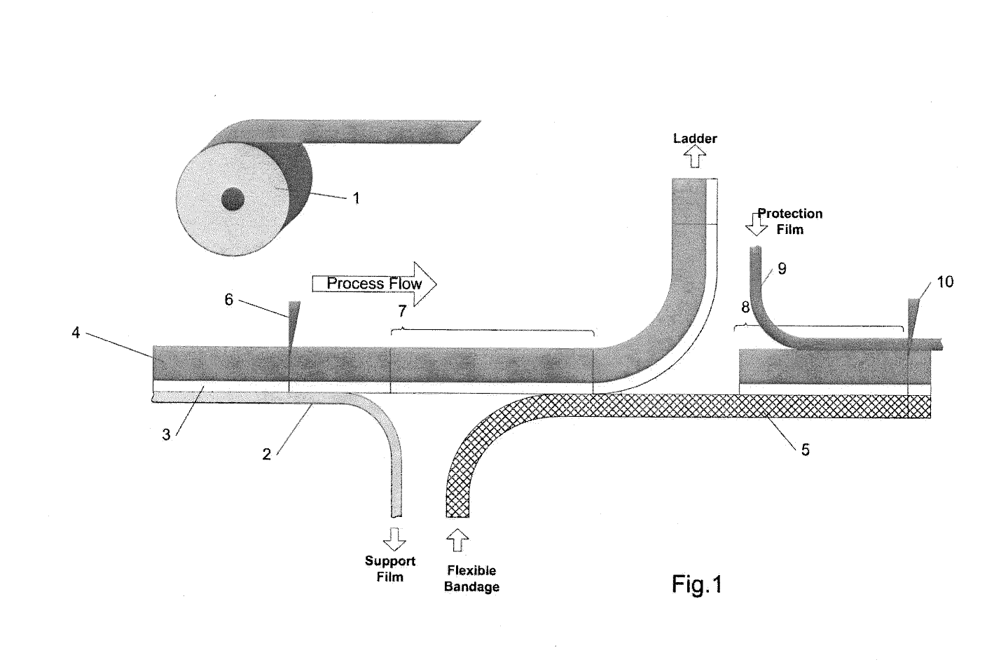

FIG. 1 shows a way of industrial manufacturing of a medical bandage of the invention. First, bulk rolls of patch (1) are manufactured. These consist of a support film (made e.g. of PET) (2), an adhesive layer (3) and an “active patch” layer (4), the latter being a matrix layer comprising the pain-relieving pharmaceutically active substance, e.g. diclofenac sodium. Bulk rolls of flexible bandage (5) are manufactured, too. It has to be mentioned that the layers (3) and (4) can also be combined to form one uniform “active patch” layer having adhesive properties.

In case that a medical bandage of the invention of which the entire inside is adhesive to the skin, is manufactured, a flexible bandage (5) will be used, on which another adhesive layer has been laminated first.

The support film (2) is progressively removed (from the patch bulk roll) as the patch frontline progresses on the line, after patch cutting (“kiss cut”, 6). The flexible bandage (5) comes in replacement to the support film (2), and only the selected patches (7,8) will be positioned on the flexible bandage (5). The adhesive layer (3) will glue the patches (7,8) on the flexible bandage (5). A continued protective film (9) comes on top of the patches and a final cut (10) defines the final shape of the medical bandage.

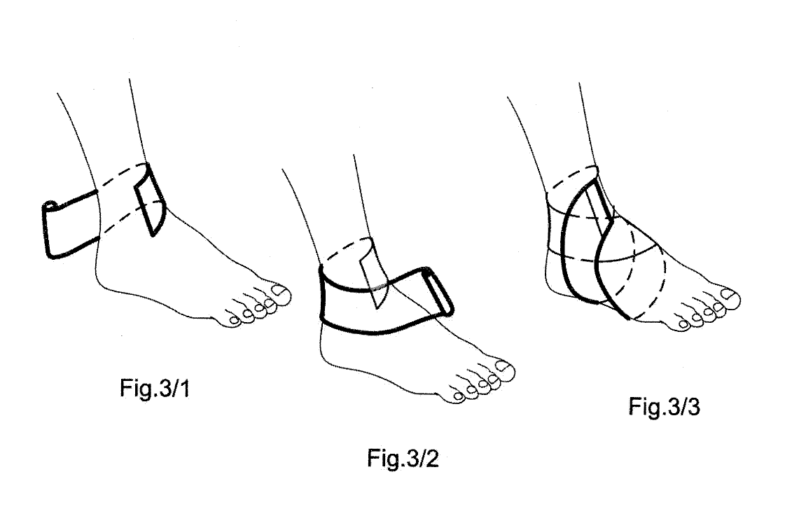

FIG. 2 shows a medical bandage (11) comprising three diclofenac patches (12a,12b,12c) for the treatment of ankle sprain. The dimensions are selected such that it will fit most bodies from teenagers (age limit according to country regulations) to adults. For adjusting and closing the medical bandage, the lower surface of the end of the bandage (13) fixes itself on the external surface of its beginning (14). The bandage material is flexible, resistant, elastic and will maintain body warmth. Patches (12a,12b,12c) are placed on the medical bandage to match the body surface close to tissues involved in pain: ligaments, tendons and joint capsules. Use and correct positioning of the bandage to ensure correct position, treatment and support are further explained in FIG. 3.

Patch width (x1, 50 mm) is smaller than bandage width (y1, 70 mm) to ensure protection of the patches (12a,12b,12c) and continued warmth. The medial ankle patch (12a) comes first on the bandage, at 50 mm from the beginning, 70 mm long. The lateral ankle patch (12b) comes next at 200 mm from the bandage beginning and is also 70 mm long. Last comes the arch-of-the foot patch (12c), starting at 350 mm from the beginning, and 220 mm long.

FIG. 3 shows the correct positioning of a bandage comprising three diclofenac patches around the ankle and foot for the treatment of ankle sprain. The steps are as follows: (3/1) Position the medical bandage beginning (14) in front of the ankle towards the medial face of the ankle. Thus the medial ankle patch (12a) is on the ankle. (3/2) Turn around the ankle towards the top of the foot. Thus the lateral ankle patch (12b) comes on the lateral face of the ankle. (3/3) Turn within the arch of the foot. Thus, the end of the bandage (13) overlaps the beginning of it (14).

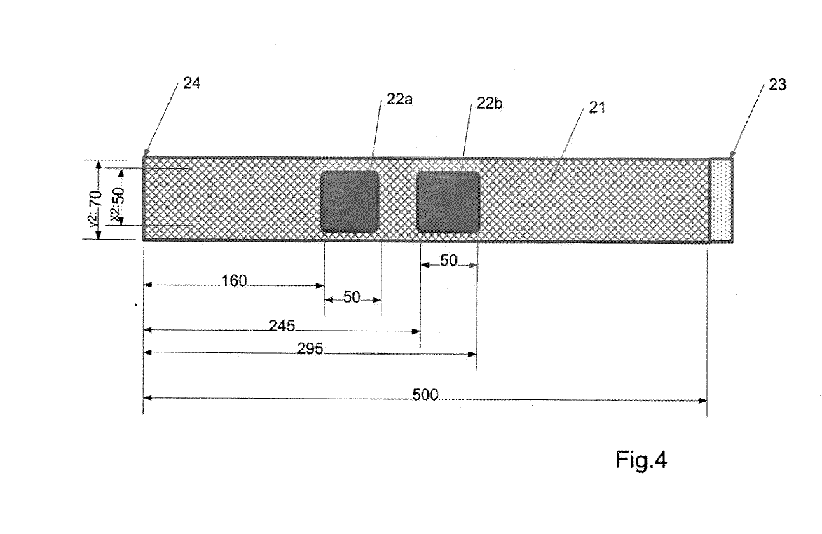

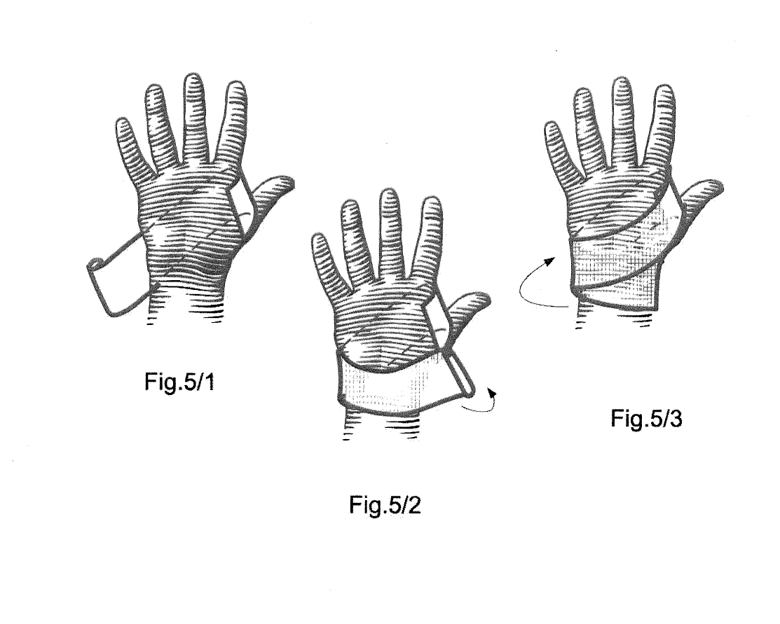

FIG. 4 shows a bandage (21) comprising two diclofenac patches (22a,22b) for the treatment of wrist pain. The dimensions are selected such that it will fit most bodies from teenagers (age limit according to country regulations) to adults. For adjusting and closing the medical bandage, the lower surface of the end of the bandage (23) fixes itself on the external surface of its beginning (24). The bandage material is flexible, resistant, elastic and will maintain body warmth. Patches (22a,22b) are placed on the medical bandage to match the body surface close to tissues involved in pain: ligaments, tendons and joint capsules. Use and correct positioning of the bandage to ensure correct position, treatment and support are further explained in FIG. 5.

Patch width (x2, 50 mm) is smaller than bandage width (y2, 70 mm) to ensure protection of the patches (22a,22b) and continued warmth. The ventral wrist patch (22a) comes first on the bandage, at 160 mm from the beginning, 50 mm long. The dorsal wrist patch (22b) comes next at 245 mm from the bandage beginning and is also 50 mm long.

FIG. 5 shows the correct positioning of a bandage comprising two diclofenac patches for the treatment of wrist pain. The steps are as follows: (5/1) Position the bandage beginning between thumb and index towards the dorsal face of the hand. (5/2) Turn around the wrist toward the ventral face of the wrist. The ventral wrist patch (22a) comes on the ventral face of the wrist. (5/3) Turn around the wrist, the dorsal wrist patch (22b) comes in place. Turn again and the bandage crosses the palm. The end of the bandage (23) overlaps the beginning of it (24).

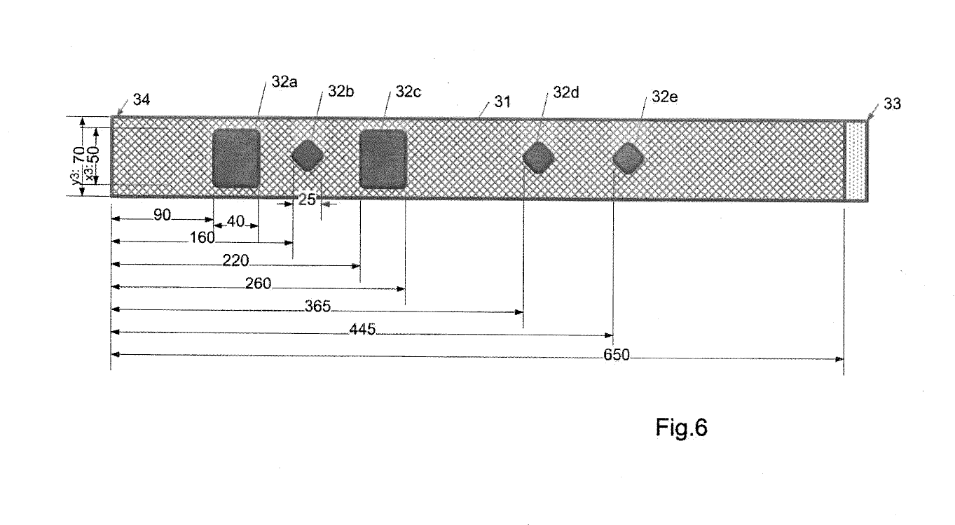

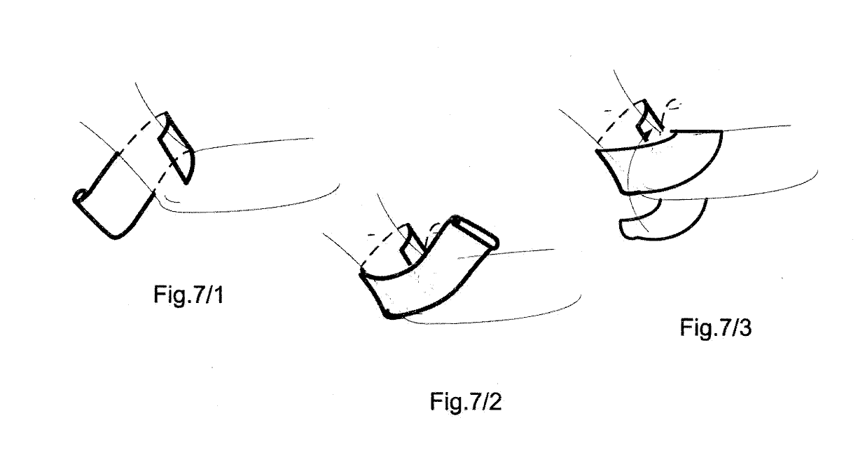

FIG. 6 shows a medical bandage (31) comprising five diclofenac patches (32a,32b,32c,32d,32e) for the treatment of elbow pain. The dimensions are selected such that it will fit most bodies from teenagers (age limit according to country regulations) to adults. For adjusting and closing the medical bandage, the lower surface of the end of the bandage (33) fixes itself on the external surface of its beginning (34). The bandage material is flexible, resistant, elastic and will maintain body warmth. Patches (32a,32b,32c,32d,32e) are placed on the medical bandage to match the body surface close to tissues involved in pain: ligaments, tendons and joint capsules. Use and correct positioning of the bandage to ensure correct position, treatment and support are further explained in FIG. 7.

Patch width (x3, 50 mm) is smaller than bandage width (y3, 70 mm) to ensure protection of the patches (32a,32b,32c,32d,32e) and continued warmth. The lateral epicondyle patch (32a) comes first on the bandage, at 90 mm from the beginning, 40 mm long, followed with the small oleocranon patch (32b).

“Small patches”, here and in the following, can be of any shape, e.g. a rhombus (lozenge) with a ca. 25 mm diagonal, or a square with ca. 25 mm side length, or e.g. a circle of ca. 25 mm diameter.

The medial epicondyle patch (32c) comes next at 220 mm from the bandage beginning and is also 40 mm long. Forearm medial patch (32d) is next, which is a small patch [25 mm rhombus (lozenge)] placed at 365 mm from the beginning. Last is the forearm lateral patch (32e), likewise a 25 mm rhombus (lozenge), starting at 445 mm from the beginning.

FIG. 7 shows the correct positioning of a bandage comprising five diclofenac patches for the treatment of elbow pain. The steps are as follows: (7/1) Position the bandage beginning (34) on medial face of the elbow, towards upper and lateral face of the arm. (7/2) Turn around the arm, on medial face of the arm cross the elbow toward the upper and lateral face of the forearm. (7/3) Turn around the forearm, on the medial face of the forearm cross again the internal face of elbow, overlapping the beginning of the bandage (34).

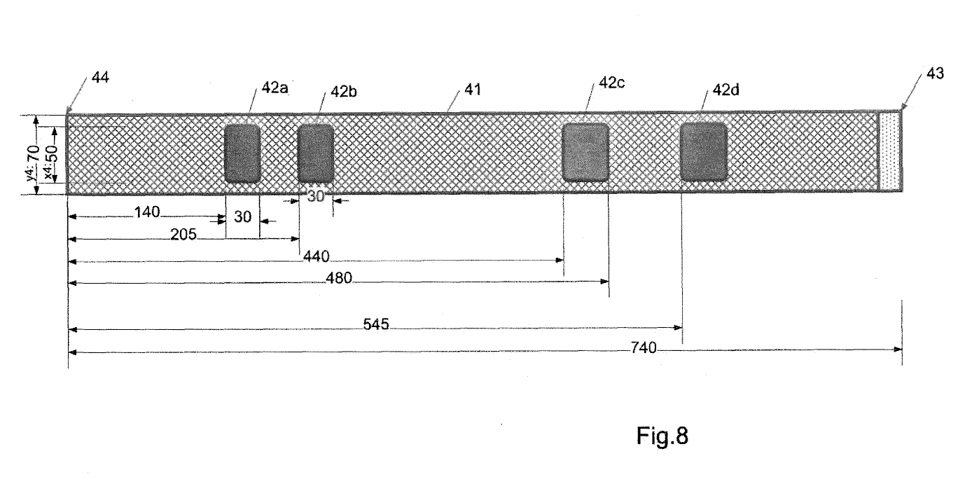

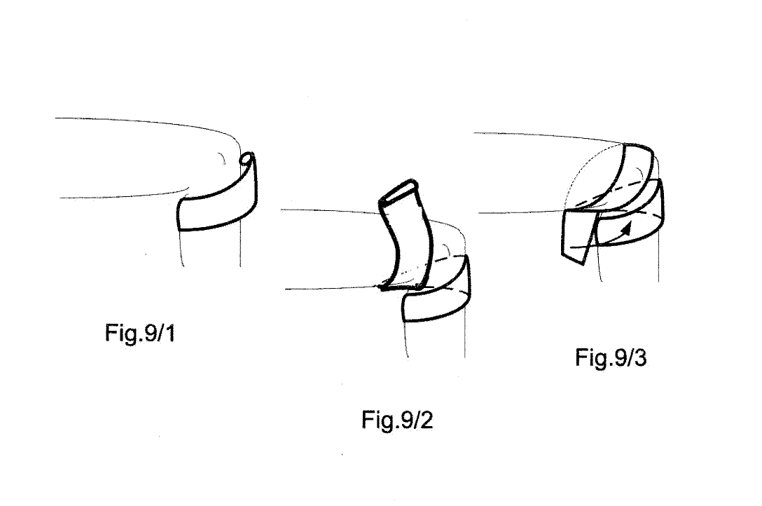

FIG. 8 shows a medical bandage (41) comprising four diclofenac patches (42a,42b,42c,42d) for the treatment of knee pain. The dimensions are selected such that it will fit most bodies from teenagers (age limit according to country regulations) to adults. For adjusting and closing the medical bandage, the lower surface of the end of the bandage (43) fixes itself on the external surface of its beginning (44). The bandage material is flexible, resistant, elastic and will maintain body warmth. Patches (42a,42b,42c,42d) are placed on the medical bandage to match the body surface close to tissues involved in pain: ligaments, tendons and joint capsules. Use and correct positioning of the bandage to ensure correct position, treatment and support are further explained in FIG. 9.

Patch width (x4, 50 mm) is smaller than bandage width (y4, 70 mm) to ensure protection of the patches and continued warmth. The medial patellar patch (42a) comes first on the bandage, at 140 mm from the beginning, 30 mm long, followed with the lateral patellar patch (42b) at 205 mm from the beginning. The knee medial patch (42c) comes next at 440 mm from the beginning and is 40 mm long. The knee lateral patch (42d) is next placed 545 mm from the beginning, 40 mm long.

FIG. 9 shows the correct positioning of a bandage comprising four diclofenac patches for the treatment of knee pain. The steps are as follows: (9/1) Position the bandage beginning (44) on medial face of the leg, towards front and lateral face of the leg. (9/2) Turn behind the knee, towards medial face of the thigh, then toward the upper face of the knee. (9/3) Turn around the thigh, when below cross behind the knee overlapping the beginning of the bandage (44).

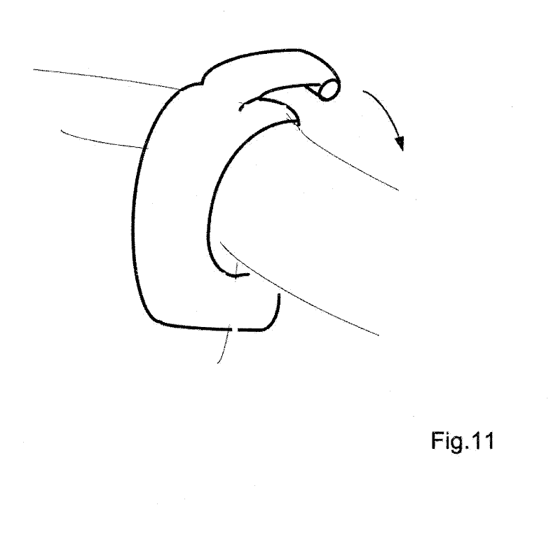

FIG. 10 shows a medical bandage (51) comprising two diclofenac patches (52a,52b) for the treatment of shoulder pain. The dimensions are selected such that it will fit most bodies from teenagers (age limit according to country regulations) to adults. For adjusting and closing the medical bandage, the lower surface of the end of the bandage (53) fixes itself on the external surface of its beginning (54). The bandage material is flexible, resistant, elastic and will maintain body warmth. Patches (52a,52b) are placed on the medical bandage to match the body surface close to tissues involved in pain: ligaments, tendons and joint capsules. Use and correct positioning of the bandage to ensure correct position, treatment and support are further explained in FIG. 11.

Patch width (x5, 50 mm) is smaller than bandage width (y5, 70 mm) to ensure protection of the patch and continued warmth. To ensure correct positioning, a hole (55) allows to put the arm through the bandage. The ventral shoulder patch (52a) comes first on the bandage, at 100 mm from the beginning, 80 mm long, followed with the dorsal shoulder patch (52b) at 220 mm from the beginning, also 80 mm long.

FIG. 11 shows the correct positioning of a bandage comprising two diclofenac patches for the treatment of shoulder pain. The steps are as follows: (11/1) Position the bandage, arm in the hole, free band vertically towards the ceiling. (11/2) Turn behind the shoulder, and down. Overlap the beginning of the bandage (54) below the shoulder.

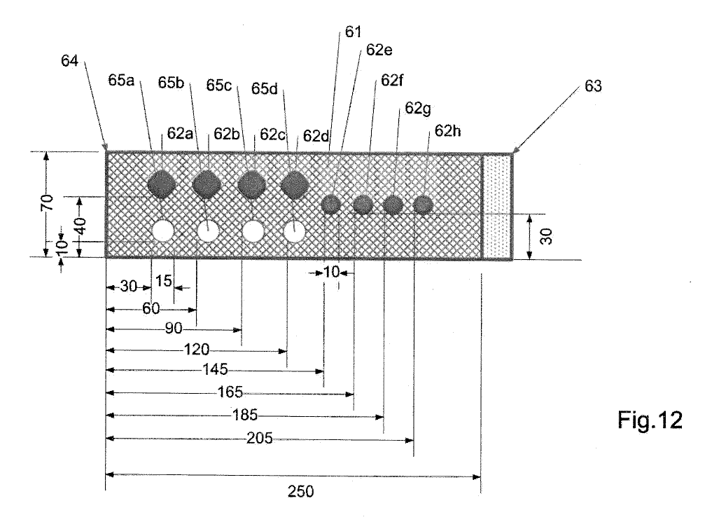

FIG. 12 shows a medical bandage (61) comprising eight diclofenac patches (62a,62b,62c,62d,62e,62f,62g,62h) for the treatment of finger pain. The dimensions are selected such that it will fit most bodies from teenagers (age limit according to country regulations) to adults. For adjusting and closing the medical bandage, the lower surface of the end of the bandage (63) fixes itself on the external surface of its beginning (64). The bandage material is flexible, resistant, elastic and will maintain body warmth. Patches (62a,62b,62c,62d,62e,62f,62g,62h) are placed on the medical bandage to match the body surface close to tissues involved in pain: ligaments, tendons and joint capsules.

Patch width is much smaller than bandage width to ensure protection of the patch and continued warmth. To ensure correct positioning, four holes (65a,65b,65c,65d) every 30 mm allow to put all fingers (except thumb) through the bandage. The dorsal patches (62a-d) are placed above the holes and will naturally be placed on the dorsal joints. At 145 mm from the bandage beginning start the smaller patches for the palm face (62e-h) of the finger joints.

It should be mention here, that it is also possible, e.g. for reasons of more efficient manufacture, to combine two or more individual patches to one. For example, in the patch of FIG. 12, in can be beneficial for manufacturing reasons to combine patches (62a) and (62b) to one patch, likewise e.g. (62c) and (62d) together, or (62e) and (620 together, or (62g) and (62h) together, or (62a), (62b), (62c) and (62d) together, or (62e), (620, (62g) and (62h) together.

To use and ensure correct positioning of the bandage to ensure correct position the bandage, start with the little finger in hole 1 (65a) if treating the right hand, but start with index finger in hole 1 (65a) if treating the left hand. Continue with other fingers into holes (65b), (65c) and (65d). Turn around the hand, cover the upper part of the joints and overlap the beginning of the bandage (64).

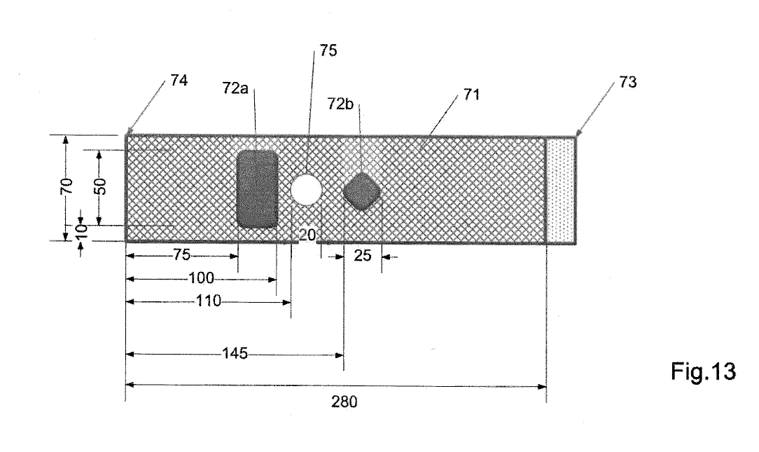

FIG. 13 shows a bandage (71) comprising two diclofenac patches (72a,72b) for the treatment of thumb pain. The dimensions are selected such that it will fit most bodies from teenagers (age limit according to country regulations) to adults. For adjusting and closing the medical bandage, the lower surface of the end of the bandage (73) fixes itself on the external surface of its beginning (74). The bandage material is flexible, resistant, elastic and will maintain body warmth. Patches (72a,72b) are placed on the medical bandage to match the body surface close to tissues involved in pain: ligaments, tendons and joint capsules.

Patch width (x7, 50 mm) is smaller than bandage width (y7, 70 mm) to ensure protection of the patch and continued warmth. To ensure correct positioning, a hole (75) at 110 mm from the bandage beginning allows to put the thumb through the bandage. The palm patch (72a) is placed 75 mm from the bandage beginning, and is 25 mm long. The dorsal thumb patch (72b),which is a small patch [25 mm rhombus (lozenge], follows at 145 mm from the bandage beginning.

To use and ensure correct positioning of the bandage to ensure correct position the bandage, start on the palm below the little finger, place the thumb in the hole (75), turn to the dorsal face of the end, and overlap the beginning of the bandage (74).

The fixation of the end of the bandage e.g. —(13), (23), (33), (43), (53), (63) or (73)—to its respective beginning—e.g. (14), (24), (34), (44), (54), (64) or (74)—can be accomplished in various ways in a manner per se. For example, mechanical means, e.g. clamps, can be used. In another embodiment, both the end of the bandage and its beginning are configured to each include an adhesive zone, which are fit together. As adhesive material comes into consideration e.g. a polyurethane, a polyacrylic adhesive, an EVA adhesive or a block elastomer such as REBS, SIS or SBS. In a further embodiment, namely in case that the adhesive material used is able to stick to the bandage material itself, it is sufficient to include an adhesive zone at either of the end of the bandage and or its beginning.

The beneficial properties of the medical bandages of the present invention are verified as follows:

The positioning of the medical bandage of Example 1 (see also FIG. 2) was tested on the ankle of people of different size—from teenagers to 110 kg weighing adults. The medical bandage adapted nicely to all of them, providing the patches at the appropriate places as intended.

Tests are run with a medical bandage according to Example 1 (see also FIG. 2) with smaller and bigger individuals—from teenagers to 110 kg weighing adults—who, are suffering from ankle sprain. Fast and reliable pain relief is observed in the group of people being treated with the medical bandage of Example 1.

The manufacture of the medical bandages of the present invention is effected in a manner known per se, for example as described in the examples below.

Typically, medical bandages of this type are produced by cutting out blanks from sheet-like material, for example from polyamide, foams (for example polyether foam), or woven fabrics of different kind including nylon or elastic fibers; or made of a guide from aluminium or molded materials such as polypropylene and fixing material. The anatomically adapted shape is achieved by the shape of the blanks or darts, for example using gusseting, and the subsequent joining together of the blanks, as is also customary in clothing.

The following examples are intended to illustrate the invention.

### EXAMPLE 1

The manufacture of a medical bandage comprising three diclofenac sodium-containing patches for the treatment of ankle sprain (see FIG. 2) is described above in connection with the explanations given for FIG. 1.

A bulk roll of patch is manufactured by laminating a matrix composition as disclosed in WO 02/22109 A2 Example 1, consisting of diclofenac sodium, styrene-isoprene-styrene copolymer, aliphatic hydrocarbon resin, oleic acid and isopropyl myristate, onto a support film made of PET.

### EXAMPLE 2

A medical bandage comprising two diclofenac sodium-containing patches for the treatment of wrist pain (see FIG. 4) is manufactured in an analogous manner as described in Example 1.

### EXAMPLE 3

A medical bandage comprising five diclofenac sodium-containing patches for the treatment of elbow pain (see FIG. 6) is manufactured in an analogous manner as described in Example 1.

### EXAMPLE 4

A medical bandage comprising four diclofenac sodium-containing patches for the treatment of knee pain (see FIG. 8) is manufactured in an analogous manner as described in Example 1.

### EXAMPLE 5

A medical bandage comprising two diclofenac sodium-containing patches and a hole for the arm for the treatment of shoulder pain (see FIG. 10) is manufactured in an analogous manner as described in Example 1.

### EXAMPLE 6

A medical bandage comprising eight diclofenac sodium-containing patches for the treatment of finger pain (see FIG. 12) is manufactured in an analogous manner as described in Example 1.

### EXAMPLE 7

A medical bandage comprising two diclofenac sodium-containing patches and a hole for the thumb for the treatment of thumb pain (see FIG. 13) is manufactured in an analogous manner as described in Example 1.

1. A medical bandage, which comprises at least two transdermal patches, wherein the at least two transdermal patches are attached to the inside of said medical bandage thus positioned to contact the patient's skin upon application of said bandage, and wherein said at least two transdermal patches each comprise a pain-relieving pharmaceutically active substance, and wherein said pain-relieving pharmaceutically active substance is selected from the group consisting of non-steroidal anti-inflammatory drugs, local anesthetics and counter irritants.

2. A medical bandage according to claim 1 wherein the at least two transdermal patches are positioned around said limb or joint such that upon application of said medical bandage the pain-relieving pharmaceutically active substance will penetrate into said limb or joint from at least two different spots close to the area that causes pain.

3. A medical bandage according to claim 1, wherein the inside thereof is adhesive to the skin.

4. A medical bandage according to claim 1, which is adapted to the treatment of ankle pain, wrist pain, knee pain, elbow pain, shoulder pain or finger pain.

5. A medical bandage according to claim 4, which medical bandage is adapted to the treatment of ankle sprain, knee sprain, knee osteoarthritis, tennis elbow, elbow osteoarthritis, finger sprain or finger osteoarthritis.

6. A medical bandage according to claim 2, which has a length of 500-800 mm, a width of 40-100 mm, and comprises a transdermal patch for the medial ankle of 40-100 mm length and 30-90 mm width, a second transdermal patch for the lateral ankle of 40-100 mm length and 30-90 mm width and a third transdermal patch for the arch-of-the-foot ankle of 80-180 mm length and 30-90 mm width.

7. A medical bandage according to claim 2, which has a length of 350-700 mm, a width of 40-100 mm, and comprises a transdermal patch for the ventral wrist of 30-80 mm length and 30-90 mm width, a second transdermal patch for the dorsal wrist of 30-80 mm length and 30-90 mm width.

8. A medical bandage according to claim 2, which has a length of 500-800 mm, a width of 40-100 mm, and comprises a transdermal patch for the lateral epicondyle of 25-70 mm length and 30-90 mm width, a second transdermal patch for the oleocranon being a small patch as defined hereinbefore, a third transdermal patch for the medial epicondyle of 25-70 mm length and 30-90 mm width, a fourth transdermal patch for the medial forearm being a small patch as defined hereinbefore, and a fifth transdermal patch for the lateral forearm being a small patch as defined hereinbefore.

9. A medical bandage according to claim 2, which has a length of 500-1100 mm, a width of 40-100 mm, and comprises a transdermal patch for the medial patella of 20-50 mm length and 30-90 mm width, a second transdermal patch for the lateral patella of 20-50 mm length and 30-90 mm width, a third transdermal patch for the medial knee of 25-70 mm length and 30-90 mm width, and a fourth transdermal patch for the lateral knee of 25-70 mm length and 30-90 mm width.

10. A medical bandage according to claim 2, which has an overall length of 500-800 mm and consists of two parts which are firmly connected, namely (a) the “hole part” and the “bandage part” (as shown in FIG. 10); wherein said hole part has a length of 150-230 mm, a width of 170-230 mm, comprises a hole which is circular and has a diameter of 70-150 mm and further comprises a transdermal patch for the ventral shoulder of 50-110 mm length and 30-90 mm width; wherein said “bandage part” has a length of 300-700 mm, a width of 40-100 mm, and comprises a transdermal patch for the dorsal shoulder of 50-110 mm length and 30-90 mm width.

11. A medical bandage according to claim 2, wherein the pain-relieving pharmaceutically active substance is diclofenac or a pharmaceutically acceptable salt thereof.

12. A medical bandage according to claim 2, which provides said pain-relieving pharmaceutically active substance at specific locations but does not allow drug permeation from the entire bandage application surface.

13. A medical bandage according to claim 1 which includes instructions in the form of one or more drawings, on how to correctly apply said medical bandage to the intended joint or limb, wherein said instructions are printed on the outside of said medical bandage.

14. A medical bandage according to claim 13, wherein said instructions indicate a starting point where to begin with the application of the medical bandage, provide information as to how to progress with the application of the medical bandage, and provide instructions on how to finally affix the bandage.

15. A process for treatment of a human patient comprising applying a medical bandage, which bandage comprises at least two transdermal patches, wherein the at least two transdermal patches are attached to the inside of said medical bandage and contact the patient's skin upon application of the bandage, wherein said at least two transdermal patches each comprise a pain-relieving pharmaceutically active substance which treats pain occurring in the limbs or joints of the patient.

16. (canceled)
